(19) United States
(12) Patent Application Publication
(10) Pub. No.: US 2016/0263212 A1
(43) Pub. Date: Sep. 15, 2016

(54) COMPOSITIONS AND METHODS FOR TOLERIZING THE IMMUNE SYSTEM TO ALLERGENS

(71) Applicant: Antera Therapeutics Inc., Boston, MA (US)

(72) Inventors: Clarence Friedman, Cambridge, MA (US); Vinidhra Mani, Boston, MA (US)

(21) Appl. No.: 15/051,458

(22) Filed: Feb. 23, 2016

Related U.S. Application Data
(60) Provisional application No. 62/119,757, filed on Feb. 23, 2015.

(51) Int. Cl. A61K 39/35; A61K 31/355; A61K 31/375; A61K 31/593; A61K 31/203

(52) U.S. Cl. CPC A61K 39/35; A61K 31/593; A61K 31/203; A61K 31/375; A61K 31/355; A61K 2039/542

(57) ABSTRACT

Compositions and methods can be used for tolerizing the immune system. The compositions can be physiologically acceptable and can include any of a wide variety of allergens that are designed to be administered in escalating doses to, for example, an infant. The compositions can include other active ingredients (e.g., one or more of a steroid, vitamin, mineral, vasodilator, hormone, decongestant, anticholinergic agent, leukotriene inhibitor, immunomodulator, mast cell stabilizer, expectorant, immune suppressant, anti-histamine, or anti-inflammatory agent) and/or a carrier.

### CROSS-REFERENCE TO RELATED APPLICATIONS

This application claims the benefit of the filing date of U.S. provisional application No. 62/119,757, which was filed Feb. 23, 2015, which is hereby incorporated by reference in its entirety.

### FIELD OF THE INVENTION

The present invention relates to compositions and methods that can be employed to tolerize the immune system and thereby lower, if not eliminate, an individual's risk of developing an allergy or the incidence of allergy within a population. The present invention relates to compositions and methods that can prevent, or reduce the risk of developing, an allergy (e.g., food allergy to peanut, shellfish, tree nut, soy, wheat, dairy, egg, citrus fruit, kiwi, banana, berry, or balsalm or environmental allergy to dust mite, animal dander, bee, pollen, ragweed, or mold, and the like).

### BACKGROUND OF THE INVENTION

The immune system is a complex system in which numerous cell types protect the body from invading pathogens. As such, there have been many studies directed to how the immune system distinguishes “self” from “non-self” and how the immune system both protects us and contributes to autoimmune disease and cancer.

Generally, the immune system does not generate a significant reaction against substances regularly encountered in the environment, such as pollen, or substances within food. This may be due to an immune tolerance mechanism that, when impaired, results in a specific immune reaction against an allergen. These allergic reactions can be severe and are sometimes fatal. When an allergic reaction does occur, it begins after an allergen has crossed an epithelial barrier and activates effector cells, for instance, in the gut lamina propria. During the early phase of a specific immune response, immunoglobulins of the E class (IgE) are produced and subsequently bind Fc receptors on the surfaces of mast cells and basophils. Upon binding and crosslinking the IgE-allergen complexes, these cells release histamine and cytokines that recruit inflammatory cells into the nasal passages and upper respiratory tract. The influx of many cell types, such as eosinophils, macrophages, lymphocytes, neutrophils and platelets, subsequently produces inflammation and amplifies the initial immune response. This can further trigger the release of more inflammatory cells.

There are a number of purported risk factors for allergy development, including family history of allergy (Koplin et al., Int. J. Environ. Res. Public Health 10(11):5364-5377, 2013), existing atopic disease (Branum et al., NCHS data brief, no 10. Hyattsville, Md.: National Center for Health Statistics, 2008; Flohr et al., J. Invest. Dermatol. 134(2):345-50, 2013), caesarian section birth (Younus et al., J. Allergy Clin. Immunol. 131.2, 2013), heavy antibiotic use (Love et al., J. Allergy Clin. Immunol. 131(2):AB331 (Abstract #L14), 2013) and antigen-specific serum IgE (van Veen et al., Clin. Transl. Allergy 3:34, 2013).

Over the last several years, the number of people suffering from allergies has increased dramatically. The theories as to why this has occurred include better hygiene, which has negatively modified the gut microbiome, changing genetic profiles, increased pollution (leading to over-sensitization), changing food processing methods, and changing diets that have affected the way the immune system develops. To date, there is no strong consensus regarding these theories, and treating allergic reactions remains challenging. There has been some success with devices to rapidly administer epinephrine and there are methods aimed at de-sensitizing already affected individuals with, for example, oral immunotherapies, but there are still no methods for tolerizing the immune system such that allergies do not develop or such that the severity of the reaction is lessened.

At least one published study suggests that altering the time at which children are introduced to allergens could affect the probability that they will develop allergies. In 2008, a study of two populations of children, one living in Israel and one living in the United Kingdom, demonstrated that the children living in Israel exhibited a 10-fold lower incidence of peanut allergies. The study hypothesized that the incidence was lower in Israeli children because foods containing peanuts are more prevalent in their country and they were exposed to those foods at a younger age (DuToit et al., J. Allergy Clin. Immunol. 122(5):984-991, 2008). On the other hand, most literature to date recommends avoidance rather than early introduction of allergens. For example, Boyce et al. (J. Allergy Clin. Immunol. 126(60):S1-S8, 2010) noted that “[t]here are no therapies available to prevent or treat food allergy: the only prevention option for the patient is to avoid the food allergen, and treatment involves the management of symptoms as they appear.” Zeiger (Pediatrics, 111(6):1662-1671, 2003) writes, “The AAP [American Academy of Pediatrics] and the European group agree generally on their recommendations for the treatment of the infant and young child confirmed with food allergy (Table 2). The causal food must be avoided.”

### SUMMARY OF THE INVENTION

In one aspect, the present invention features compositions for tolerizing the immune system in a manner that reduces an individual's risk of developing an allergy. The compositions can be physiologically acceptable and non-naturally occurring and can include an allergen, a biologically active additive and/or a carrier. Any type of allergen or any combination of allergens can be included in the present compositions and used in the present methods. For example, the allergen can be a food allergen (e.g., a tree nut, egg, soy, dairy, shellfish, wheat, citrus fruit, kiwi, banana, berry, or balsalm allergen), an environmental allergen (e.g., a house dust mite, animal dander, bee, pollen, ragweed, or mold allergen), or any combination thereof. Food and environmental allergens can be contained within a composition, such as a food product, that is incorporated into the present compositions. For example, the composition containing the allergen can be produced by manipulating a natural product containing the allergen. For example, the allergen can be incorporated within crushed, ground, or pureed nuts, seeds, plant tissue, dairy products, fish, shellfish, and the like. The allergen can also be contained within more highly processed compositions, such as butters, oils, and flours, extracts and purifications. For example, where the allergen is a peanut allergen, it can be contained within peanut butter, peanut oil, or peanut flour that is combined with a biologically active additive and/or a carrier. This extends to all food allergens. For example, where the allergen is a sesame allergen, it may be contained within sesame butter, sesame oil, or sesame flour.

As the biologically active additive, the compositions can include one or more of the following: a steroid (e.g., a cholesterol, progestin, androgen, estrogen, or corticosteroid), a vitamin (e.g., one or more of vitamin A, a B vitamin (e.g., vitamin B6), vitamin C, vitamin D, or vitamin E), a mineral (e.g., calcium or zinc), a vasodilator, a hormone (e.g., epinephrine), a decongestant, an anticholinergic agent, a leukotriene inhibitor, an immunomodulator, a mast cell stabilizer, an expectorant, an immune suppressant, an anti-histamine (e.g., etirizine, chlorpheniramine, diphenhydramine, fexofenadine, hydroxyzine, or loratadine), or an anti-inflammatory agent (e.g., plant tissue, an extract, or a compound from Boswellia (a genus of trees in the order Sapindales), Curcuma longa (the source of turmeric and turmeric root, or a member of the nettle genus Urtica), or any combination thereof. Pluralities of one or more of these types of additives may be included. For example, the present compositions can include more than one type of anti-inflammatory agent (e.g. more than one anti-inflammatory steroid and/or more than one plant extract with anti-inflammatory properties), more than one type of vitamin (e.g., a combination of vitamins A, C, D, and E), more than one type of mineral, etc. One or more of these additives can also be specifically excluded from a composition of the invention. For example, the compositions may exclude a particular steroid, vitamin, and/or mineral or may exclude all steroids, vitamins, and/or minerals.

Any of the compositions can further include a carrier as described below (e.g., a preservative that facilitates a shelf life of at least one month and preferably up to 12 months).

In the methods of the invention, a plurality of allergens (i.e., two or more (e.g., 2-10)) can be introduced to an individual in a scheduled manner (e.g., sequentially, as described below).

In particular embodiments, the compositions of the method are liquid suspensions containing one or more allergens (e.g., a peanut flour suspension) in an aqueous solution or in oil (e.g., safflower oil or rice oil). The compositions can be contained (e.g., within a tube, vial, squeeze-packet) and packaged within the kits described herein, together with instructions for use. The composition can be contained in single dose, e.g., single dose tube. A kit can be setup to include multiple doses. In one embodiment, the compositions include about 0.1-20.0 g (e.g., about 1, 2, 2.5, 5, 7.5, 10, 12, or 15 grams) of the allergen (e.g., peanut protein). In one embodiment, the composition includes <140 g per kit, e.g. in 5 g per vial increments. The allergen may be synthesized or purified or, as noted, may be contained within a composition such as a food product (e.g., a butter, oil, or flour) that includes other substances in addition to the allergen. The quantities we describe are meant to apply to either the allergen per se (e.g., a synthesized or purified peanut allergen) or to the composition of which that allergen is a part. For example, where a composition of the invention includes gram quantities of an allergen (e.g., about 0.1-20 g), the weight values may be of the allergen per se (e.g., a synthesized or purified peanut allergen) or of a composition in which that allergen is present (e.g., a butter, oil, or flour containing the allergen). In certain embodiments, a unit dosage form of the present compositions will include at least 0.5 g of the allergen or of a food product including the allergen. The volume of the unit dosage forms can vary from about 50 microliters-20 mL, or 2-20 mL (e.g., about 4 ml), and the amount of the allergen can be reduced based upon a risk category of a subject, discussed in further detail below (e.g., to about 1/10^{th} (e.g., to about 0.1 g) as a first dose for high-risk infants.

In another aspect, the invention features kits comprising a plurality of physiologically acceptable compositions, as described herein. The compositions within the kits can include an allergen and, in some embodiments, one of the compositions within the plurality (e.g., one of 5, one of 7, or one of 28 unit doses) can include a different amount of the allergen. Similarly, the unit dosage forms within a packaged kit can include different allergens. The kits can further include instructions for use and/or paraphernalia for administering the composition to an individual).

In another aspect, the invention features single dose containers (e.g., vials, tubes, or sachets) comprising an effective amount of a composition as described herein. More generally, the compositions may be formulated in unit dosage forms. In one embodiment, the unit dosage form is a liquid preparation (e.g., an aqueous, water-based preparation or an oil-based preparation formulated as a dispersion, solution, suspension or emulsion) of the allergen (or allergens) with a carrier and, optionally, one or more of the additives described herein. The liquid preparation (e.g., a suspension) can include oil (e.g., any physiologically acceptable oil, such as safflower oil, rice oil, peanut oil, a tree oil, mineral oil, gingelly or sesame oil, and coconut oil). Regardless of the precise formulation or packaging, the containers can include a tamper-resistant seal. Regardless of the precise make up, the compositions of the invention can include less than 2% water (w/v or v/v).

In another aspect, the invention features a food product (e.g., a baby food, snack food, or formula) comprising an effective amount of a composition as described herein. The present compositions would be mixed with the food product (e.g., a food or beverage such as formula or cereal appropriate for consumption by an infant or young child).

In another aspect, the invention features methods of reducing an individual's risk of developing an allergy. An advantage of these methods is that they can include risk stratification (i.e., assessing an individual's level of risk of developing an allergy as a high, moderate, or low risk). The methods can also include making an allergy diagnosis and/or recommending a treatment for reducing the risk of allergy development in an individual (e.g., a human infant). More specifically, the methods can include a stratification step and/or a treatment step. In the stratification step, the individual is assessed in order to determine their risk of being allergic to, or developing an allergy to, any given allergen. This assessment can include non-invasive steps, such as asking questions, and/or invasive steps such as administering a small amount of an allergen to the skin. The treatment steps can include administering to the individual a plurality of physiologically acceptable compositions at distinct points in time, and the composition administered at a first distinct point in time may include a lower amount of an allergen than a composition administered at a second distinct point in time. In the stratification and treatment methods, the individual can be an infant who has a parent or an older sibling (related by blood) with an allergy; an individual having an IgE level against whole peanut or against one or more of Ara h 1-Ara h 9 of less than about 1 kU/L (2.4 ng/mL (e.g., 0.1-0.5 (e.g., 0.35) kU/L)); an individual who tests negative for pin prick test as determined by a wheal size of less than about 4 mm (where histamine is 4 mm (e.g., about 1 mm) in diameter; a newborn baby or infant under 18 months of age (e.g., an infant between 4 and 6 months of age, with treatment continuing for about 24 months after it has begun); or an individual having one or more of a genetic predisposition to allergy, eczema, egg allergy, a parent with an allergy that has been alleviated, or a parent with an existing allergy. Although peanut is provided throughout the application as one exemplary embodiment, the invention is not necessarily limited to peanut and, in various embodiments, is directed to multiple allergens. Assessing an individual's risk using such information is described further below. The individual may meet one or more of these criteria and may also fail to meet one or more (but not all) of these criteria. For example, the individual may have no eczema and/or no egg allergy. Similarly, wheal size, which we include in our stratification protocol by determining whether the wheal measures greater than or less than 4 mm, provides a means for assessing a patient and determining the manner in which they may be treated. Neither wheal size nor any other assessments that can be made to stratify patients are strict indicators for exclusion from treatment. Generally, upon administration over time, the amount(s) of the allergen(s) in subsequent administrations increases, and the increase may be linear or logarithmic.

In the compositions and methods described herein, the first composition can comprise 0.1 mg 20.0 g of allergen or the composition containing the allergen (e.g., peanut flour); the compositions can be administered at 2-730 (e.g., about 2-365 or 2-120) distinct points in time; the time between successive administrations can be at least or about multiple daily doses or one day, one week, or one month; and the compositions can be administered orally (e.g., by swallowing a liquid formulation) or parenterally (e.g., transdermally, topically, or by administration to a mucous membrane). The kits and any packaged versions of the present compositions can include varying numbers of containers in which the present compositions are conveniently aliquoted and labeled to accommodate such dosing schedules. For example, the kits or any packaged versions of the present compositions can include an initial, low dosage form of the composition alone and/or a certain supply (e.g., a five-day, seven-day, or monthly supply) of the compositions.

As used herein “allergenic substance” is a substance that includes an allergen (e.g., allergenic protein) but may also include one or more other components that are not allergenic per se. For example, “allergenic substance” can be a protein derived from the substance (e.g., including essentially allergenic protein(s) or, in some cases including allergenic protein(s) along with one or more other components derived from the allergenic substance). For example peanut flour can be deemed an “allergenic substance” because it includes one or more allergenic proteins (e.g., Ara-h1, etc.).
In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is peanut protein, and wherein the target amount is 5/7 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is shellfish protein, and wherein the target amount is 1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is tree nut protein, and wherein the target amount is 12/7 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is soy protein, and wherein the target amount is 6/7 g and the variance is 20%;
B. a vitamin supplement comprising two or more of:

400 IU±20% vitamin D3;

750 IU±20% vitamin A;

35 mg±20% vitamin C;

5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is wheat protein, and wherein the target amount is 1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is dairy protein, and wherein the target amount is 1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is egg protein, and wherein the target amount is 1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is peanut protein, and wherein the target amount is 5 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is shellfish protein, and wherein the target amount is 5 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is tree nut protein, and wherein the target amount is 5 mg-2 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is soy protein, and wherein the target amount is 5 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is wheat protein, and wherein the target amount is 3 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is dairy protein, and wherein the target amount is 3 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a composition comprising one or more dosages of an admixture, wherein each dosage of the admixture includes the following:
A. a quantity of allergenic substance, wherein the quantity is defined as a target amount plus/minus a variance, wherein the allergenic substance is egg protein, and wherein the target amount is 3 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a method of allergy prevention comprising:
A. selecting a subject without an allergic reaction to an allergenic substance, wherein the allergenic substance is peanut protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 5 mg-1 g and the variance is 20%; ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is shellfish protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 5 mg-1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is tree nut protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 5 mg-2 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is soy protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 5 mg-1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is wheat protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 3 mg-1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is dairy protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 3 mg-1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is egg protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 3 mg-1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In still further aspects the invention provides a composition as described above the composition of any one of claims 1-7, wherein the composition comprises a single unit dose of the admixture.
   In still further aspects the invention provides a composition as described above the composition of any one of claims 1-7, wherein the variance is 15%.
   In still further aspects the invention provides a composition as described above the composition of any one of claims 1-7, wherein the variance is 10%.
   In still further aspects the invention provides a composition as described above the composition of any one of claims 1-7, wherein the variance is 5%.
   In still further aspects the invention provides a composition as described above the composition of any one of claims 1-7, wherein each the variance is 1%.
   In still further aspects the invention provides a composition as described above the composition of any one of claims 1-12, wherein the vitamin supplement comprises two or more of:
   400 IU±15% vitamin D3;
   750 IU±15% vitamin A;
   35 mg±15% vitamin C; and
   5 IU±15% vitamin E.
      In still further aspects the invention provides a composition as described above the composition of any one of claims 1-12, wherein the vitamin supplement comprises two or more of:
   400 IU±10% vitamin D3;
   750 IU±10% vitamin A;
   35 mg±10% vitamin C; and
   5 IU±10% vitamin E.
      In still further aspects the invention provides a composition as described above the composition of any one of claims 1-12, wherein the vitamin supplement comprises two or more of:
   400 IU±5% vitamin D3;
   750 IU±5% vitamin A;
   35 mg±5% vitamin C; and
   5 IU±5% vitamin E.
      In still further aspects the invention provides a composition as described above the composition of any one of claims 1-12, wherein the vitamin supplement comprises two or more of:
   400 IU vitamin D3;
   750 IU vitamin A;
   35 mg vitamin C; and
   5 IU vitamin E.
      In still further aspects the invention provides a composition as described above the composition of any one of claims 1-13, wherein the physiologically acceptable carrier comprises a vegetable oil.
      In still further aspects the invention provides a composition as described above the composition of any one of claims 1-13, wherein the physiologically acceptable carrier comprises safflower oil.
      In still further aspects the invention provides a composition as described above the composition of any one of claims 1-15, wherein the physiologically acceptable carrier comprises rice oil. In still further aspects the invention provides a composition as described above the composition of any one of claims 1-19, wherein each dosage comprises 50 microliters-20 mL of the admixture.
      In still further aspects the invention provides a composition as described above the composition of any one of claims 1-20, wherein the vitamin supplement comprises two of the vitamin D3, vitamin A, vitamin C, and vitamin E.
      In still further aspects the invention provides a composition as described above the composition of any one of claims 1-20, wherein the vitamin supplement comprises three of the vitamin D3, vitamin A, vitamin C, and vitamin E.
      In still further aspects the invention provides a composition as described above the composition of any one of claims 1-20, wherein the vitamin supplement comprises all four of the vitamin D3, vitamin A, vitamin C, and vitamin E.
      In still further aspects the invention provides a composition as described above the composition of any one of claims 1-7, wherein:

A. the composition comprises a single 50 microliters-20 mL unit dose of the admixture;

B. the variance is 5%;

C. component (B) comprises:
400 IU±5% vitamin D3,
750 IU±5% vitamin A,
35 mg±5% vitamin C,
5 IU±5% vitamin E; and
D. component (C) comprises a vegetable oil.
   In still further aspects the invention provides a composition as described above the composition of any one of claims 1-24, formulated for oral administration.
   In various aspects, the invention features a method of allergy prevention comprising:
A. selecting a subject without an allergic reaction to an allergenic substance, wherein the allergenic substance is peanut protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 5/7 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is shellfish protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is tree nut protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 12/7 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is soy protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 6/7 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is wheat protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is dairy protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a method of allergy prevention comprising:
A. selecting a subject without an allergic reaction to an allergenic substance, wherein the allergenic substance is egg protein;
B. repeatedly administering a dosage of an admixture to the subject, wherein the dosage of the admixture includes the following:
i. a quantity of the allergenic substance, wherein the quantity is defined as a target amount plus/minus a variance, and wherein the target amount is 1 g and the variance is 20%;
ii. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In still further aspects the invention provides a composition as described above the selecting step includes selecting, as the subject, an infant.
   In still further aspects the invention provides a composition as described above the infant is 3-12 months old at a time of selection.
   In still further aspects the invention provides a composition as described above the infant is 3-6 months old at a time of selection.
   In still further aspects the invention provides a composition as described above the infant is 3-4 months old at a time of selection.
   In still further aspects the invention provides a composition as described above step (B) includes administering the dosage regularly.
   In still further aspects the invention provides a composition as described above step (B) includes administering the dosage at least weekly.
   In still further aspects the invention provides a composition as described above step (B) includes administering the dosage at least daily.
   In still further aspects the invention provides a composition as described above step (B) includes administering the dosage for at least two months.
   In still further aspects the invention provides a composition as described above step (B) includes administering the dosage for at least three months.
   In still further aspects the invention provides a composition as described above step (B) includes administering the dosage for at least six months.
   In still further aspects the invention provides a composition as described above step (B) includes administering the dosage for at least twelve months.
   In still further aspects the invention provides a composition as described above step (B) includes administering the dosage for at least twenty-four months.
   In still further aspects the invention provides a composition as described above the variance is one of the following: 15%, 10%, 5%, or 1%.
   In still further aspects the invention provides a composition as described above the admixture comprises a vitamin supplement selected from:
A. Two or more of the following: 400 IU±15% vitamin D3, 750 IU±15% vitamin A, 35 mg±15% vitamin C, and 5 IU±15% vitamin E;
B. Two or more of the following: 400 IU±10% vitamin D3, 750 IU±10% vitamin A, 35 mg±10% vitamin C, and 5 IU±10% vitamin E;
C. Two or more of the following: 400 IU±5% vitamin D3, 750 IU±5% vitamin A, 35 mg±5% vitamin C, and 5 IU±5% vitamin E;
D. Two or more of the following: 400 IU vitamin D3, 750 IU vitamin A, 35 mg vitamin C, and 5 IU vitamin E.
   In still further aspects the invention provides a composition as described above the physiologically acceptable carrier comprises a vegetable oil.
   In still further aspects the invention provides a composition as described above the physiologically acceptable carrier comprises any of rice oil and safflower oil.
   In still further aspects the invention provides a composition as described above each dosage comprises 50 microliters-20 ml of the admixture.
   In still further aspects the invention provides a composition as described above the vitamin supplement comprises three or four of the vitamin D3, vitamin A, vitamin C, and vitamin E.
   In still further aspects the invention provides a composition as described above the dosage is administered orally.
   In various aspects, the invention features a method of allergy prevention comprising:
A. selecting a subject without an allergic reaction to a predetermined allergenic substance; and
B. repeatedly administering a dosage of the admixture according to any of the aspects disclosed herein to the subject, thereby preventing or reducing a risk that the subject will develop an allergy to the predetermined allergenic substance.
   In still further aspects the invention provides a composition as described above the dosage is administered orally.
   In various aspects, the invention features a composition comprising one or more dosages of an admixture, wherein each dosage of the admixture includes the following:
A. two or more of:
i. a quantity of allergenic substance, wherein the quantity is defined as a target amount plus/minus a variance, wherein the allergenic substance is peanut protein, and wherein the target amount is 5/7 g and the variance is 20%;
ii. a quantity of allergenic substance, wherein the quantity is defined as a target amount plus/minus a variance, wherein the allergenic substance is shellfish protein, and wherein the target amount is 1 g and the variance is 20%;
iii. a quantity of allergenic substance, wherein the quantity is defined as a target amount plus/minus a variance, wherein the allergenic substance is tree nut protein, and wherein the target amount is 12/7 g and the variance is 20%;
iv. a quantity of allergenic substance, wherein the quantity is defined as a target amount plus/minus a variance, wherein the allergenic substance is soy protein, and wherein the target amount is 6/7 g and the variance is 20%;
v. a quantity of allergenic substance, wherein the quantity is defined as a target amount plus/minus a variance, wherein the allergenic substance is wheat protein, and wherein the target amount is 1 g and the variance is 20%;
vi. a quantity of allergenic substance, wherein the quantity is defined as a target amount plus/minus a variance, wherein the allergenic substance is dairy protein, and wherein the target amount is 1 g and the variance is 20%;
vii. a quantity of allergenic substance, wherein the quantity is defined as a target amount plus/minus a variance, wherein the allergenic substance is egg protein, and wherein the target amount is 1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a method of allergy prevention comprising:
A. selecting a subject without an allergic reaction to an allergenic substance, wherein the allergenic substance is shellfish protein;
B. repeatedly administering a dosage of an admixture to the subject, wherein the dosage of the admixture includes the following:
   a. two or more of:
i. a quantity of allergenic substance, wherein the quantity is defined as a target amount plus/minus a variance, wherein the allergenic substance is peanut protein, and wherein the target amount is 5/7 g and the variance is 20%;
ii. a quantity of allergenic substance, wherein the quantity is defined as a target amount plus/minus a variance, wherein the allergenic substance is shellfish protein, and wherein the target amount is 1 g and the variance is 20%;
iii. a quantity of allergenic substance, wherein the quantity is defined as a target amount plus/minus a variance, wherein the allergenic substance is tree nut protein, and wherein the target amount is 12/7 g and the variance is 20%;
iv. a quantity of allergenic substance, wherein the quantity is defined as a target amount plus/minus a variance, wherein the allergenic substance is soy protein, and wherein the target amount is 6/7 g and the variance is 20%;
v. a quantity of allergenic substance, wherein the quantity is defined as a target amount plus/minus a variance, wherein the allergenic substance is wheat protein, and wherein the target amount is 1 g and the variance is 20%;
vi. a quantity of allergenic substance, wherein the quantity is defined as a target amount plus/minus a variance, wherein the allergenic substance is dairy protein, and wherein the target amount is 1 g and the variance is 20%;
vii. a quantity of allergenic substance, wherein the quantity is defined as a target amount plus/minus a variance, wherein the allergenic substance is egg protein, and wherein the target amount is 1 g and the variance is 20%;
   b. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In various aspects, the invention features a composition comprising one or more dosages of an admixture, wherein each dosage of the admixture includes the following:
A. two or more of:
i. a quantity of allergenic substance, wherein the quantity is defined as a target amount plus/minus a variance, wherein the allergenic substance is peanut protein, and wherein the target amount is 5 mg-1 g and the variance is 20%;
ii. a quantity of allergenic substance, wherein the quantity is defined as a target amount plus/minus a variance, wherein the allergenic substance is shellfish protein, and wherein the target amount is 5 mg-1 g and the variance is 20%;
iii. a quantity of allergenic substance, wherein the quantity is defined as a target amount plus/minus a variance, wherein the allergenic substance is tree nut protein, and wherein the target amount is 5 mg-2 g and the variance is 20%;
iv. a quantity of allergenic substance, wherein the quantity is defined as a target amount plus/minus a variance, wherein the allergenic substance is soy protein, and wherein the target amount is 5 mg-1 g and the variance is 20%;
v. a quantity of allergenic substance, wherein the quantity is defined as a target amount plus/minus a variance, wherein the allergenic substance is wheat protein, and wherein the target amount is 3 mg-1 g and the variance is 20%;
vi. a quantity of allergenic substance, wherein the quantity is defined as a target amount plus/minus a variance, wherein the allergenic substance is dairy protein, and wherein the target amount is 3 mg-1 g and the variance is 20%;
vii. a quantity of allergenic substance, wherein the quantity is defined as a target amount plus/minus a variance, wherein the allergenic substance is egg protein, and wherein the target amount is 3 mg-1 g and the variance is 20%;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier.
   In various aspects, the invention features a method of allergy prevention comprising:
A. selecting a subject without an allergic reaction to an allergenic substance, wherein the allergenic substance is shellfish protein;
B. repeatedly administering a dosage of an admixture to the subject, wherein the dosage of the admixture includes the following:
   a. two or more of:
i. a quantity of allergenic substance, wherein the quantity is defined as a target amount plus/minus a variance, wherein the allergenic substance is peanut protein, and wherein the target amount is 5 mg-1 g and the variance is 20%;
ii. a quantity of allergenic substance, wherein the quantity is defined as a target amount plus/minus a variance, wherein the allergenic substance is shellfish protein, and wherein the target amount is 5 mg-1 g and the variance is 20%;
iii. a quantity of allergenic substance, wherein the quantity is defined as a target amount plus/minus a variance, wherein the allergenic substance is tree nut protein, and wherein the target amount is 5 mg-2 g and the variance is 20%;
iv. a quantity of allergenic substance, wherein the quantity is defined as a target amount plus/minus a variance, wherein the allergenic substance is soy protein, and wherein the target amount is 5 mg-1 g and the variance is 20%;
v. a quantity of allergenic substance, wherein the quantity is defined as a target amount plus/minus a variance, wherein the allergenic substance is wheat protein, and wherein the target amount is 3 mg-1 g and the variance is 20%;
vi. a quantity of allergenic substance, wherein the quantity is defined as a target amount plus/minus a variance, wherein the allergenic substance is dairy protein, and wherein the target amount is 3 mg-1 g and the variance is 20%;
vii. a quantity of allergenic substance, wherein the quantity is defined as a target amount plus/minus a variance, wherein the allergenic substance is egg protein, and wherein the target amount is 3 mg-1 g and the variance is 20%;
   b. a vitamin supplement comprising two or more of:
   400 IU±20% vitamin D3;
   750 IU±20% vitamin A;
   35 mg±20% vitamin C;
   5 IU±20% vitamin E; and
C. thereby, preventing or reducing a risk that the subject will develop an allergy to the allergenic substance.
   In still further aspects the invention the admixture or composition comprises <2% water.

The term “about” is used herein to indicate that a value includes an inherent variation of error for the device or the method being employed to determine the value or plus-or-minus 10% of the stated value, whichever is greater.

An “allergen” is any substance that elicits an unwanted immune response in some individuals and may be a naturally or non-naturally occurring substance or a fragment or other variant thereof. The fragments and variants may or may not include all or other known properties of the naturally or non-naturally occurring allergen but will be allergenic and useful in tolerizing the immune system as described herein to reduce an individual's risk of developing an allergy or to reduce the incidence of an allergy in a population. The fragment or other variant can be a portion of a synthetic or natural allergen (e.g., a portion of an allergenic peptide, glycan, or lipid, or any combination thereof) or an identified epitope (e.g., Ara h I-III of peanut) that elicits an immune response. To confirm the suitability of a fragment or variant for inclusion in the compositions described herein, one can determine whether the fragment or variant elicits mast cell degranulation in an in vitro assay.

An “effective amount” of an allergen is an amount sufficient to reduce the risk or incidence of developing a cognate allergy without adverse side effects when the allergen is administered as described herein. An effective amount of an allergen can be as described herein and may vary at certain points in time (e.g., over the course of an escalating dosage regimen) and further as selected by one of ordinary skill in the art depending on a particular individual recipient or type of individual (e.g., an underweight individual). Thus, an effective amount can vary from individual to another, due to variation in metabolism, age, weight, sex, diet, the general condition of the subject, the allergy being targeted, and the judgment of the prescribing physician.

A composition is “physiologically acceptable” when it is non-toxic as formulated for administration to a population of human recipients (e.g., infants).

In case of doubt, the present methods, which rely on early exposure for reducing the risk of allergy development are distinct from, and can be performed to the exclusion of, immunotherapy for allergy treatment. Because of the regimen followed to treat an individual (and, where included, the stratification steps carried out prior to treatment), the methods are also distinct from the simple act of randomly consuming foods, beverages, or other products that may include allergens. Similarly, we specify that the present compositions are non-naturally occurring in order to emphasize that the compositions of the invention are not simply natural products or foods and beverages currently made from natural products. For example, the compositions of the invention are not simply nuts, seeds, grains, grain plants, other plants (e.g., ragweed), soy, fish, shellfish, milk, eggs, other dairy products (e.g., cream), other animal products, or fruits; are not foods and beverages currently made from these natural products; and are not balsalm, pollen, rubber, mold, or dust per se. These products (e.g. nuts), foods and beverages containing them (e.g., peanut butter), and/or allergens within them can be incorporated into the present compositions but they do not, in and of themselves, constitute the present compositions.

### BRIEF DESCRIPTION OF THE DRAWINGS

FIG. 1 is a schematic representation of a treatment regime in which five allergens (P=peanut; S=soy; TN=tree nut; Sh=shellfish; and E=environmental) are introduced on the days indicated over a period of two months. An advantage of regimes such as this is that they can be carried out with a limited number of manufacturing units. The subject/patient is initially administered a dose every three days, and a consistent daily dosage of all allergens (here, the five types of allergens listed above) is achieved by the end of the second month of treatment. The total amount of protein administered daily may not exceed about 12 grams. No allergen is introduced within 3 days of another one.

FIG. 2 is a table listing the ingredients included in an exemplary 4 ml dose of a composition of the invention.

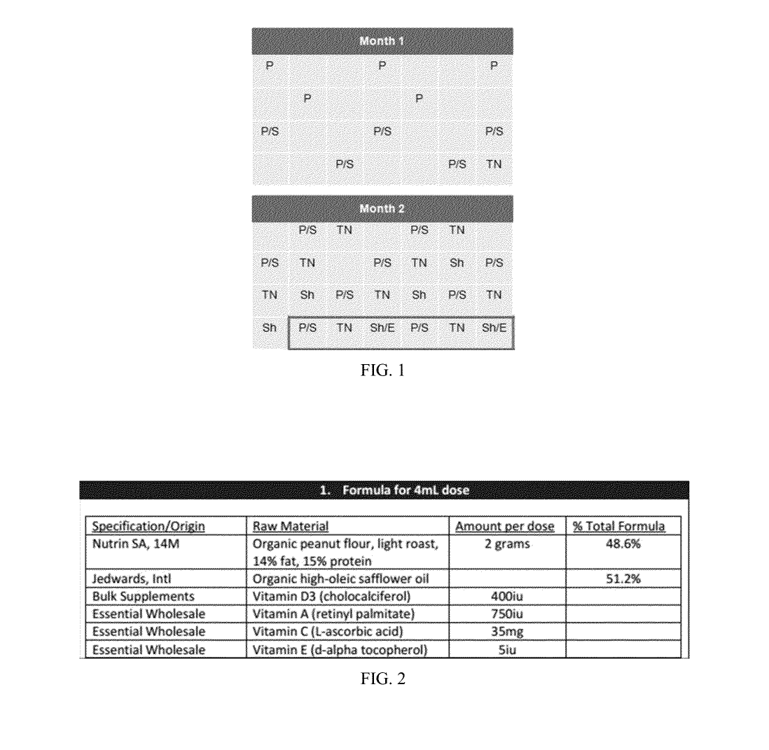

### DETAILED DESCRIPTION OF THE INVENTION

The present invention features, inter alia, compositions and methods for tolerizing the immune system, preferably in infants, to reduce the risk or incidence of allergies in later life. The compositions will include at least one allergen, such as an allergen from nuts (e.g., peanuts or tree nuts, such as almonds, brazil nuts, cashews, chestnuts, hazelnuts, pistachios, and walnuts), seeds (e.g., sunflower, pumpkin, or sesame seeds), grains and grain-bearing plants (e.g., wheat and wheat plants), other plants (e.g., ragweed), soy (e.g., soybeans or soybean plants), fish and shellfish, dairy products (e.g., milk and eggs) and other animal products (e.g., bee venom), fruits (e.g., citrus fruits, kiwi, bananas, and berries), balsalm, and pollen. The allergen can also be an antibiotic or contained within or associated with a rubber product or environmental allergen such as mold (e.g., mold spores) or dust. The allergen can also be an antibiotic or contained within or associated with a rubber product or environmental allergen such as mold (e.g., mold spores) or dust. In specific embodiments, the allergen can be Ara h 1-9 (from the peanut), Cas s 5 or Cas s 8 (from the chestnut, Castanea sativa), Cor a 1, 1.0401, 1.0402, 1.0403, 1.0404, Cor a 2, Cor a 8, Cor a 9, Cor a 11 (from the hazelnut, Corylus avellana), Ber e 1 or Ber e 2 (from the brazil nut Bertholletia excelsa), Jug n 1 or Jug n 2 (from the black walnut, Juglans nigra), Jug r 1-4 (from the English walnut, Juglans regia), Ana o 1, 1.0101, 1.0102, or Ana o 2 (from the cashew nut Anacardium occidentale), tropomyosin (from shellfish), ovomucoid (also known as Gal d 1), ovalbumin, ovotransferrin, lysozyme, livetin, apovitillin, and phosvitin (from eggs); casein (from milk), and albumins, globulins, prolamins, glutelins, gliadins, and glutenins (from wheat). Powders, solutions, suspensions, and the like made from the source of an allergen or a fractionated part or extract thereof can also be incorporated into the present compositions as the allergen. Further, the compositions described herein that are administered to a patient may be classified as Food For Special Dietary Use (e.g., a composition classified by a regulatory agency such as the Food and Drug Administration as a Food For Special Dietary Use formulated to safely reduce the risk of peanut allergy development in infants), food for special dietary use, conventional foods, Food For Special Dietary Use, or drug.

Biologically Active Additives:

In addition to containing one or more allergens, the compositions can also include one or more biologically active additives that may also be physiologically or pharmaceutically acceptable and/or carriers that are active or inert, in accordance with the commonly accepted understanding of pharmaceutical carriers. Biologically active additives include steroids (also referred to as steroid hormones, e.g., a corticosteroid such as cortisol), pharmaceutical drugs with corticosteroid-like effects (e.g., dexamethasone, prednisone, fludrocortisone, and hydrocortisone), vitamins (e.g., a vitamin A, vitamin B, vitamin C, vitamin D, or vitamin E), minerals, vasodilators, hormones (e.g., epinephrine), decongestants, anticholinergics, leukotriene inhibitors, immunomodulators, mast cell stabilizers, expectorants, immune suppressants, anti-histamines, anti-inflammatories, an anti-IgE antibody (e.g., omalizumab), a short- or long-acting beta agonist (e.g., albuterol/salbutamol, bambuterol, fenoterol, isoetherine, isoproterenol, levalbuterol, metaproterenol, pirbuterol, terbutaline, and tornalate), or any combination thereof. The additional active ingredient can also be a medication that augments the process of tolerizing an individual's immune system or that limits the severity of an immune reaction, including anaphylaxis, if it were to occur. Any of the additional ingredients can be naturally occurring or synthetic, and the compositions of the invention can include one or more of chlorpheniramine, vitamin A, a B vitamin (e.g., vitamin B6), vitamin C, vitamin D, vitamin E, a polyphenol, zinc, diphenhydramine, hydroxyzine, cetirizine, fexofenadine, loratadine, boswellia, turmeric root, and nettle. As one of ordinary skill in the art would recognize, active ingredients may be classified in more than one way. For example, corticosteroids may be classified as steroids, steroid hormones, or anti-inflammatory agents.

In one embodiment, a composition described herein comprises an allergen (e.g., a peanut or other allergen) in a concentration from about 0.05% to about 99% w/w or w/v, or any integer therein (e.g. 0.5%-95%; 1.0%-90%; 10%-80%; 25%-75%; or 40%-60% w/w or w/v). Similarly, in one embodiment, a composition described herein comprises a second active ingredient (e.g., an anti-inflammatory agent or vitamin) in a concentration from about 0.05% to about 99% w/w or w/v, or any integer therein (e.g. 0.5%-95%; 1.0%-90%; 10%-80%; 25%-75%; or 40%-60% w/w or w/v). First and subsequently administered compositions (e.g., second, third, fourth, fifth, and sixth compositions) can include 0.05-10,000 mg of allergen.

Carriers:

As noted, the compositions can also include at least one carrier, which we define as a moiety useful as a binder, colorant, diluent, disintegration agent, excipient, filler, hapten, lubricant, preservative, stabilizer, surfactant, suspension agent or solubilization agent, wetting agent, or a substance that masks an unpleasant flavor or smell. More specifically, the carrier can be or can include water (including any type of distilled or purified water), acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, any type of salt, including sodium chloride, tricalcium phosphate, and dipotassium phosphate, cellulose and cellulose conjugates, sugars, sodium stearoyl lactylate, carrageenan, monoglyceride, diglyceride, pregelatinized starch, and the like. If necessary, one of ordinary skill in the art can consult industry resources, such as Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). Where the carrier serves primarily as a filler, it can be a compound such as lactose, calcium carbonate, calcium phosphate, dibasic calcium phosphate, calcium sulfate, microcrystalline cellulose, cellulose powder, dextrose, dextrate, dextran, a starch, a pregelatinized starch, a sugar, such as sucrose, xylitol, lactitol, mannitol, sorbitol, a salt such as sodium chloride, polyethylene glycol, and combinations thereof.

The carrier, whether used to formulate a liquid or solid (e.g., powdered) formulation, can be used to ensure a composition (e.g., a unit dosage form) has an adequate volume (e.g., for manageable dispersal from various containers (e.g., vials) throughout the treatment regimen). Where the carrier is employed primarily as a diluents, it can be, for example, alginic acid and salts thereof, cellulose and derivatives thereof, such as carboxymethylcellulose, methylcellulose (e.g., METHOCEL® methylated cellulose), hydroxypropylmethylcellulose, hydroxyethylcellulose, hydroxypropylcellulose (e.g., KLUCEL® water soluble polymer), ethylcellulose (e.g., ETHOCEL® ethyl cellulose), microcrystalline cellulose (e.g., AVICEL® unprocessed cellulose crystallite); silicified microcrystalline cellulose; microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose, glucose, dextrose, molasses, mannitol, sorbitol, xylitol (e.g., XYLITAB® artificial sweetener), lactose (e.g., lactose monohydrate, lactose anhydrous, etc.); dicalcium phosphate; a natural or synthetic gum such as acacia, tragacanth, ghatti gum, mucilage of isapol husks, polyvinylpyrrolidone (e.g., polyvidone, Kollidon® CL vinyl polymerisate, Polyplasdone® XL chemical), larch arabogalactan, VEEGUM® magnesium aluminum silicate, polyethylene glycol, waxes, sodium alginate, starches (e.g., a natural starch such as corn starch or potato starch, a pregelatinized starch such as Colorcon (Starch 1500), National 1551 or AMIJEL® corn starch, or sodium starch glycolate such as Explotab® disintegrating agent); a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as VEEGUM® magnesium aluminum silicate; a gum such as agar, guar, locust bean, Karaya, pectin, or tragacanth; sodium starch glycolate; a natural sponge; a surfactant; a resin such as a cation-exchange resin; citrus pulp; sodium lauryl sulfate; distilled water, saline solutions, or organic solvents; and combinations thereof. Examples of useful fillers include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and combinations thereof. Examples of useful solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, cholesterol, bile salts, polyethylene glycol 200-600, glycofurol, transcutol, dimethyl isosorbide and combinations thereof.

As would be understood, any given substance employed as a carrier may serve more than one purpose, and many such ingredients can be incorporated in the present compositions provided they are compatible with the allergen(s) being formulated and the release profiles of the desired dosage forms.

Any of the compositions described herein may be formulated as a liquid (e.g., a solution (e.g., an aqueous solution) or a liquid emulsion (e.g., an oil-based emulsion)). One or more of these formulations, or combinations thereof, can be included in the kits of the invention. As used herein, a “solution” is used to refer to a liquid mixture in which the minor component (the solute) is uniformly distributed within the major component (the solvent). The compositions can also be formulated as dispersions, which are also mixtures, and may be a liquid, solid (e.g., powder) or gas in which one component (e.g., the solute) is distributed either uniformly or non-uniformly, within another component (e.g., the solvent). An emulsion is a type of liquid formulation in which small droplets of one liquid are contained in another in which it is not soluble.

The allergen or any additives with which it is administered can be isolated from natural extracts, can be synthesized, or can be contained within products (such as peanut butter).

In one embodiment, a final formulation includes a composition as described herein that has been mixed with or mixed into one or more of the following items: a food or drink (e.g., a taste-masking food such as baby formula), or a dietary supplement. Whether or not the final formulation of the composition of the invention includes one or more vitamins, it may also be mixed with a vitamin preparation prior to administration. The mixture may be prepared within days, hours, or minutes of its administration to an individual.

Sources of Allergens:

A wide variety of allergens can be formulated and administered as described herein, including those found in plants and animals as well as in non-naturally occurring materials. Sources of allergens include nuts (e.g., peanuts), grains (e.g., wheat), soy, fish and shellfish, dairy products and other animal products (e.g., bee venom). The allergens within the present compositions can be purchased from commercial suppliers or manufactured using methods known in the art. For example, compositions including allergens from peanuts may be initially sourced from raw peanuts, such as Arachis hypogaea, procured from multiple farming sources (e.g., the Golden Peanut Company, where shelled, raw peanuts are processed into 12% defatted roasted peanut flour). Compositions such as this peanut flour may be further processed (e.g., under cGMP conditions), formulated with a carrier (e.g., in a 1:1 ratio of peanut flour:carrier, w/w or w/v), and subsequently distributed into containers (e.g., sterile, single-use vials; an embodiment encompassed by the present invention). Specific allergens from peanuts and other sources are listed above.

Formulations:

The compositions described herein can be formulated for administration to an individual via oral administration. Conventional pharmacological techniques that can be used to make the present compositions include dry mixing, direct compression, milling, dry or non-aqueous granulation, wet granulation, and fusion, which may be used singly or in any combination. See, e.g., Lachman et al., The Theory and Practice of Industrial Pharmacy (1986). Other useful methods include, for example, spray drying, pan coating, melt granulation, granulation, fluidized bed spray drying or coating (e.g., Wurster coating), tangential coating, top spraying, tableting, extruding and the like. The compositions can be created using Good Manufacturing Processes, to be checked using standard quality control metrics such as spectroscopy based on known standards.

At various time points (e.g., within a set time of administering an allergen), a sample of plasma can be obtained will be stored for assessment of specific antibody levels. Total IgE and specific IgE and IgG4 can be measured by UniCAP™. Any parameters measured can be measured prior to the first administration of the allergen to establish a base line value.

In some embodiments, the composition can be formulated in a unit dosage form of less than about 5 mL (e.g., about 4.5, 4.0, 3.5, 3.0, or 2.5 mL). The formulation may include peanut flour (e.g., about 2 grams or less than 50% (e.g., about 48.6%) of the formulation). The peanut flour may be organic light roast peanut flour and may include fat (e.g., about 14% fat) and protein (e.g., 15%, 42-50% protein). Any of the allergens described herein can be formulated to include or further include organic high-oleic safflower oil (e.g., about half (e.g., about 51.2%) of the formulation). The formulation may further include vitamin D3 (cholocalciferol, e.g., 400 IU), vitamin A (retinyl palmitate, e.g., 750 IU), vitamin C (L-ascorbic acid, e.g., 35 mg), vitamin E (d-alpha tocopherol, e.g., 5 IU) or any combination thereof. Any of these formulations may be an oil-based emulsion. In some embodiments, a dose of the formulation can include up to about 20 g of a protein (e.g., about 10-20 g of a protein or any range or amount therebetween), up to about 50 g of an allergen, or a combination thereof. In a specific embodiment, a composition of the invention includes 750 IU vitamin A, 35 mg vitamin, 400 IU vitamin D, and 1 gram of protein in non-GMO, organic, whole peanut flour in an oil-based emulsion.

Any of the compositions of the invention can be formulated to have one or more of the following characteristics:

| Property | One Embodiment | Range |
|---|---|---|
| Viscosity | 100 cps | 3 cps-30,000 cps at |
| | | 20-25 C. (room temp) |
| Protein content | 1 g | 0.1-20 g |
| Fat content | 3 g | 1-20 g |
| Carbohydrate content | 0.01 g | 0-30 g |
| Water content | 2% | 0-5% |
| Smoke Point | 160 C. | 100-260 C. |
| Protein concentration | 0.25 g/mL | 0.1 g/mL-10 g/mL |

With regard to other formulations, conventional pills or tablets, including chewable formulations and Food For Special Dietary Use (e.g., FDA defined Food For Special Dietary Use), can be used when the individual is old enough to swallow such a preparation without difficulty.

The kit or package can include any type of adherence packaging, and the packaging may be designed for compliance with regimen. The kit or package can also include one or more of the following components: a tube, an extrusion bag, an oral syringe (e.g., pre-loaded), a syringe or dropper for unit doses from bottles, a baby-feeding bottle, a squirt bottle, flexible package, a teether (e.g., for an infant, a child, or an adult), a shaker for powder formulation, a packet for powder formulation. The kit or package can include one or more doses (e.g., 5 or 7) of the composition. The doses may be packaged in a same container, in multiple attached containers, or separate containers.

The kit or package can include one or more materials. The material can be any antimicrobial or non-toxic material. For example, the composition may be contained in a tube, and the coating or the material of the tube may be aluminum or polyethylene. The kit or package may include guidance for physicians or for individuals at home. Where tubes are included in the kits, they can be single use, twist-off tubes, extended twist-off nasal tubes, or neckless tubes. The material incorporated into the tubes can include an aluminum barrier or foil layered laminate web, EVOH (ethylene vinyl alcohol), and/or low density polyethylene. The tubes or any other containers within the kits can vary in size and may be between about 13-19 mm in diameter and 35-60 mm in length or 10-30 mm in diameter, 20-75 mm in length). The containers can also be colored or opaque to help ensure product stability and integrity.

In a preferred embodiment, a 4 mL dose of the composition may be contained in a container (e.g., a tube) that includes a polymer (e.g., ethene-loctene copolymer, CAS No. 26221-73-8).

The compositions within the kit may be packaged such that one part of the kit is housed separately from another. For example, a first box within the kit may include a composition intended to be administered by a physician in a medical setting (e.g., a hospital) as the initial dose in the regimen and a second box may include additional compositions intended for administration away from a medical setting (e.g., by parents at home).

Methods of Preparation:

The present invention features methods of manufacturing the compositions, including specific formulations and kits, described here. The methods may include one or more of the following steps in a process of preparing a composition for administration to an individual: (1) adding a required amount of an oil (e.g., safflower oil) for a batch at, e.g., 70°−80° F., to a vessel (e.g., a stainless steel vessel) with high mix/shearing capacity, and gradually adding the allergen (e.g., peanut flour) to the oil during mixing; (2) adding the required amount(s) of an additive/additives (e.g., vitamins) to the batch during mixing to generate a product; (3) mixing the product until there are no visible lumps (e.g., of peanut flour), discharging the smooth, mixed product into a covered secondary stainless steel vessel for packaging; (4) stirring or otherwise agitating the product during packaging to ensure even distribution of the ingredients in each dose; and (5) heating the product (e.g., to 190° F. for 30 seconds) during the filling stage to kill bacteria or prevent bacterial growth. Sterilization may occur by pasteurization. It is likely advisable to cover the secondary vessel containing product to be packaged for no longer than three hours after mixing is completed. Where vitamins C and E are added, they may be powders, and where vitamins A and D are added, they may be liquids. The method may further include one or more of the following steps: (1) adjusting the pH value of the product to better solubilize or homogenize the product; and (2) testing or verifying the killing of bacteria or prevention of bacterial growth (e.g., using a microbe challenge test).

Methods of Use:

While we may describe the methods herein as methods of treatment, any such descriptions can be equally well presented as “uses” and the invention can be claimed as the use of a composition described herein in, for example, the preparation of a medicament or in the preparation of a medicament for inducing tolerance in accordance with varying patent practices throughout the world. While the compositions described herein are intended to be administered to humans (e.g., human infants). Similarly, while the compositions of the invention are intended to be administered to very young individuals, individuals of any age can be treated as described herein, particularly if they have not be previously exposed to the allergen. For example, one can administer an allergen from insect venom (e.g., bee venom) to an adult who has never been stung or bitten by the insect in question. Thus, in various embodiments, the individual can be a newborn baby, an infant up to 18 months old (e.g., a treatment regimen as described herein can begin when the infant is about 1, 2, 4, 6, 8, 10, 12, or 15 months old), a child, adolescent, or adult (i.e., a person over 18 years old). When used to prevent the development of allergies in infants, we may refer to the present methods as constituting an early introduction platform.

While the methods can be carried out without any prior evaluation of an individual, they may also be preceded by tests or other evaluative methods (e.g., questioning and medical history review) to determine whether the individual is an especially good candidate. For example, the present methods can include an evaluative step comprising an analysis of the individual's personal or familial history of eczema or a personal or familial history of any known allergies (e.g., an allergy to eggs; sibling histories may be particularly informative). One can also consider whether the individual's parents have a former allergy that was alleviated or an allergy that still exists (or existed throughout their lifetime). One can also subject the individual to a skin prick test for an allergen (e.g., a food or airborne allergen). Typically, a skin test probe is pressed through an allergen (e.g., a commercial extract of an allergen) onto the epidermis. Positive (histamine) and negative (saline-glycerin) controls can be placed to establish that the response is not blocked and to determine if there is dermatographism, respectively. One can also evaluate the individual for a mutation to the filagren gene comprising R501X, 2284del4, 3321delA, S2554X, or 2284del4.

The methods of the present invention can be stratification methods, which can be based on the results from the evaluative step, to stratify the individual into a category (e.g., excluded from treatment, low risk, moderate risk, or high risk).

The individual can be stratified into a “low risk” group (e.g., low risk of allergy to, for example, peanuts) if the individual satisfies one or more of the following criteria: (1) no history of the allergy (or allergies) in question in the family, (2) no atopic conditions, (3) previous skin test wheal size=0 mm or RAST<0.35, and (4) prior exposure to traces of the allergen (e.g., peanuts) with no noticeable reaction. By “in the family” or “family members” we mean the blood-related siblings and parents of the individual being tested.

The individual can be stratified into a “moderate risk” group (e.g., moderate risk of allergy to, for example, peanuts) if the individual satisfies one or more of the following criteria: (1) birth by caesarian section, (2) mild, non-persistent/intermittent asthma, (3) history of the food allergy (or allergies) in the family, (4) mild eczema, (5) other mild atopic disease, and (6) heavy antibiotic use in early childhood (i.e., three or more courses of antibiotics). The severity of the individual's asthma can be assessed as described in Colice (Clin. Med. Res. 2(3):155-163, 2004), which provides the following classifications:

Classification of Asthma Severity Method of Expert Panel Report 2²⁵

| | | Nighttime | |
|---|---|---|---|
| Severity | Symptoms | Symptoms | Lung Function |
| Severe | Contional symptoms | Frequent | FEVₜ/PEF ≦50% |
| persistent | Limited physical activity | | PEF variability |
| | Frequent exacerbations | | [symbol] 30% |
| Moderate | Daily symptoms | >1/week | FEV₁ [symbol] PEF >60% |
| persistent | Daily use of short-acting | | but <80% |
| | beta-agoniss | | PEF variability |
| | Exacerbations affect | | >30% |
| | activity | | |
| | Exacerbations ≧2/week | | |
| Mild | Symptoms >2/week but | >2/month | FEV₁/PEF ≧30% |
| persistent | <1/day | | PEF variability |
| | Exacerbations may affect | | 20% to 30% |
| | activity | | |
| Mild | Symptoms ≦2/week | ≦2/month | FEV₁ [symbol] PEF ≧80% |
| intermittent | Asymptomatic between | | PEF variability |
| | exacerbations | | <20% |
| | Exacerbations brief | | |

FEV₁ [symbol] forced expiratory volume in one second;
PEF: peak expiratory flow [symbol]
indicates data missing or illegible when filed

The individual can be stratified into a “high risk” group (e.g., high risk of allergy to, for example, peanuts) if the individual satisfies one or more of the following criteria: (1) severe eczema, (2) other severe atopic conditions, and (3) a medical history including an allergic reaction. The severity of the eczema can be determined using the EASI score (Eczema Area and Severity Index) as described, for example, by Hanifin et al. (Exp. Dermatol. 10(1):11-18, 2001). As a part of the risk assessment, the individual (or the individual's parent or guardian) can complete a questionnaire. If the individual is found to have a low to medium risk, the first dose of an allergen composition can be given immediately or scheduled for an appropriate time. If the individual is found to have a high risk of developing an allergy, the individual may be subjected to a skin prick test and treatment may begin subsequently if determined sufficiently safe. For example, if the skin prick test reveals a wheal size of <4 mm (where histamine if 4 mm or more and saline is 0 mm), the patient may be allowed to continue to the first oral dose. The initial dose of the allergen composition may also be modified, depending on an individual's risk level, as follows. If the individual is found to have a low to medium risk, the first administration can consist of a normal initial dosage of a particular allergen. If the individual is found to have a high risk, the first administration can consist of a reduced (e.g., 1/10^{th} normal) initial dosage followed by a 30 minute waiting period. If there is no adverse reaction after that time (e.g., no hives or rash, no swelling, no difficulty breathing, no bleeding, and no signs of GI discomfort), the individual can be given a higher dose (e.g., a half-normal or normal initial dosage) with continued monitoring. After these initial treatments, the individual can continue treatment at home.

The individual (e.g. an infant) can be ineligible for the treatment, thereby being stratified into “excluded from treatment”, if the individual has one or more of the following pre-existing medical conditions: allergic colitis, gastroenteritis, pyloric stenosis, other gastrointestinal disorders, cardiovascular disorders, genetic disorders (e.g. Down's syndrome), severe persistent asthma or other chronic respiratory disease, autoimmune disease, immunodeficiency, premature birth, or frequent anaphylaxis.

In some embodiments, the individual may be ineligible for treatment if the treatment is contraindicated. For example, the individual may be ineligible for immediate treatment if the individual used (1) long-acting antihistamines within the past seven days, (2) short-acting antihistamines within two days, or (3) beta-2 agonists within the past 48 hours. The individual may also be ineligible if he or she received one or more vaccines within 72 hours prior to the treatment. The individual must not exhibit any of the following symptoms within 72 hours prior to treatment: non-eczema related rashes or hives, fever, acute GI symptoms (e.g. vomiting, diarrhea, and/or constipation), an acute respiratory illness, or systemic illness (e.g. influenza, rhinovirus).

The compositions described herein can be administered according to a variety of regimens, including those in which an individual receives an escalating amount of the allergen over time. For example, the methods can include administering an allergen-containing formulation from two to 100 times over the course of two days to 1-5 years (e.g., 2-5 administrations over 2 days to 2 months). While the prior statement is intended to illustrate the variability of the dosing regimen, one of ordinary skill in the art would likely avoid the extremes of administering 100 compositions in two days or two compositions in 1-5 years. In one embodiment, after an individual is given an initial dose of an allergen and experiences no serious side effects, the individual can be treated with 1, 2, 3, 4, 5, 6, 7, 8 or 9 additional escalating doses of a composition described herein. The additional escalating doses may be administered to a subject in intervals spanning days, months, or years, and the escalation may occur in a linear or non-linear manner. For example, the dosage can be increased by virtue of doubling or tripling dosages, by exponential increases, or by a combination thereof. Moreover, at any point, a given dosage may be administered more than once prior to escalation. For example, an individual may receive 2 mg of an allergen at 1-5 points in time before the dosage is increased to 6 mg (which may be given at multiple points in time prior to the next increase, if any). In one embodiment, escalating doses are administered to the individual on day 1 of treatment. For example, a subject may be administered 1, 2, or 3 doses of a composition described herein on day 1. For example, a subject may be administered 5 doses of a composition described herein in 30 minute increments on day 1. The dosages at various plateau levels may be increased by a factor of 5 (e.g., 2, 3, or 5 mg is increased to 10 mg; 1000 mg is increased to 5000 mg) or 10 (e.g., 10 mg is increased to 1000 mg). The precise dosing regimen may be varied depending on each individual's response. For example, if an individual exhibits no adverse reactions, the time frame initially planned for allergen administration can be compressed. An exemplary dosing regimen is illustrated in the table below:

| Dose # | Month/Day | Dose (mg) | Cumulative Dose (mg) |
|---|---|---|---|
| 1 | 4/1 | 2 (or 3 or 5) | 2 (or 3 or 5) |
| 2 | 4/15 | 10 | 12 |
| 3 | 5/1 | 100 | 112 |
| 4 | 5/15 | 1000 | 1112 |
| 5 | 6/1 | 5000 | 6112 |
| 6 | 6/15 | 5000 | 11112 |

In other embodiments, the method can comprise the steps of (a) administering to an individual escalating doses of 0.005 g to 5 g, e.g., 0.5 mg, 1.0 mg, 1.5 mg, 3.0 mg, and 6.0 mg in 30-minute intervals on day 1 of the treatment regimen; (b) optionally, administering to the individual a maximum tolerated dose on day 2 of the treatment regimen; and (c) administering to said subject single doses of 0.005 g to 5 g, e.g., 12 mg, 20 mg, 40 mg, 80 mg, 120 mg, 160 mg, 200 mg, 240 mg, and 300 mg in two-week intervals. The formulations described herein are administered and dosed in accordance with good medical practice, and at least the initial administration can include relatively small quantities of allergen (0.005 to 5 g/dose (e.g., 2 mg/dose)). The dosages can also be evaluated and administered based on comparisons to quantities contained in food or to amounts which the individual would otherwise likely be exposed. In any of the dosing regimens, an individual can be evaluated for the onset of systemic symptoms as evidenced by flushing, itchy skin, sneezing, runny nose, perceived heat, general discomfort, and agitation or anxiety.

As described above, during the treatment, the administration of an allergen may be repeated over a length of time. The length of the treatment may be 3-30 months (e.g. at least or about three months for low risk individuals; at least or about six months for moderate risk individuals; and at least or about 12-24 months for high risk individuals).

During the treatment, the individual may take one of the following actions if the individual misses one or more of the doses: (1) continue dosing at home and monitor for warning signs or (2) schedule a first administration within 2 weeks (e.g., following the eligibility tests). The individual may be in or after the first 3 months of the treatment. The individual may also be stratified as “low risk”, “moderate risk”, or “high-risk” as described above.

For example, the individual may continue treatment at home with monitoring for warning signs if the individual is in the first three (3) months of the treatment and is: (1) a “low risk” individual who missed 10 or fewer consecutive doses, (2) a “moderate risk” individual who missed 10 or fewer consecutive doses, or (3) a “high risk” individual who missed three (3) or fewer consecutive doses.

The individual may be scheduled for a first administration within two weeks if the individual is in the first 3 months of the treatment and is: (1) a “low risk” individual who missed over 10 consecutive doses, (2) a “moderate risk” individual who missed over 10 consecutive doses, or (3) a “high risk” individual who missed over 3 consecutive doses.

In other examples, the individual may continue dosing at home and monitor for warning signs if the individual is after the first 3 months of the treatment and is: (1) a “low risk” individual who missed 15 or fewer consecutive doses, (2) a “moderate risk” individual who missed 15 or fewer consecutive doses, or (3) a “high risk” individual who missed 3 or fewer consecutive doses.

The individual may schedule a first administration within 2 weeks if the individual is after the first 3 months of the treatment and is: (1) a “low risk” individual who missed over 15 consecutive doses, (2) a “moderate risk” individual who missed over 15 consecutive doses, or (3) a “high risk” individual who missed over 3 consecutive doses.

An individual treated as described herein may exhibit a decreased anaphylactic reaction, relating to a decrease in clinical symptoms following treatment of symptoms associated with exposure to an anaphylactic allergen, which can involve exposure via cutaneous, respiratory, gastrointestinal, and mucosal (e.g., ocular, nasal, and aural) surfaces or a subcutaneous injection (e.g., via a bee sting) following treatment.

Any reaction, whether occurring in the clinic or at home, that requires two or more doses of epinephrine will halt further dose escalation for that individual. Maintenance of the last tolerated dose could be continued.

As noted above, the present compositions encompass multi-allergen products, and the methods of the invention encompass the administration of those products. Treatment with multiple allergens can also be staged over time, with a first allergen being administered at a first point in time, a second allergen being administered at a second, later point in time, and so on. The amounts of the allergens can be determined by the protein in a serving size (where applicable), and the order of their introduction, while variable, can be determined by the typical age for allergy onset and allergen exposure. For example, infants are often exposed to foods containing nuts before they are exposed to foods containing shellfish. The intervals between the introduction of the allergens (e.g., between the first and second allergen; the second and third allergen; and so forth) can vary and may be about one week, two weeks, or three weeks. An exemplary schedule is as follows:

| Allergen | Protein content | First introduced |
|---|---|---|
| Peanut | 5 g | First week of treatment |
| Soy | 6 g | Third week of treatment |
| Tree Nut | 12 g | Fifth week of treatment |
| Shellfish | 7 g | Seventh week of treatment |
| Environmental | 4 g | Ninth week of treatment |

Daily, weekly, and monthly doses as well as daily volumes can be selected from the following table.

| Allergen | Daily | Weekly | Monthly | Volume (daily) |
|---|---|---|---|---|
| Peanut | 5 mg-1 g | 5 mg-7 g | 5 mg-35 g | 0.05-20 mL |
| Soy | 5 mg-1 g | 5 mg-7 g | 5 mg-35 g | 0.05-20 mL |
| Shellfish | 5 mg-1 g | 5 mg-7 g | 5 mg-35 g | 0.05-20 mL |
| Tree Nut | 5 mg-2 g | 5 mg-14 g | 5 mg-70 g | 0.05-20 mL |
| Environmental | 3 mg-1 g | 3 mg-7 g | 3 mg-35 g | 0.05-20 mL |
| Egg | 3 mg-1 g | 3 mg-7 g | 3 mg-35 g | 0.05-20 mL |
| Dairy | 3 mg-1 g | 3 mg-7 g | 3 mg-35 g | 0.05-20 mL |
| Wheat | 3 mg-1 g | 3 mg-7 g | 3 mg-35 g | 0.05-20 mL |

As noted above, the present compositions can include two active ingredients; an allergen (or combination of allergens) and a second active ingredient such as a steroid. In the context of the present methods, the two active ingredients can be formulated and administered separately (e.g., by the same route of administration at different times or by different routes of administration at about the same time or sequentially). The determination of the mode of administration and the advisability of administration, where possible, in the same formulation, is well within the knowledge of the skilled clinician. The initial administration can be made according to established protocols known in the art, and then, based upon the observed effects, the dosage, modes of administration and times of administration can be modified by the skilled clinician. Accordingly, in the kits of the invention, the vials provided may include the allergen and a second active ingredient separately. For sequential administrations, the time period between the multiple administration steps may range from a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval. “Bioavailability” refers to the percentage of the weight of allergen(s) dosed that is delivered into the general circulation of the animal or human being studied. The total exposure (AUC_{((0-&infin;))}) of a drug when administered intravenously is usually defined as 100% Bioavailable (F %). “Oral bioavailability” refers to the extent to which the allergen(s) are absorbed into the general circulation when the composition is taken orally as compared to parenteral administration (e.g., intravenous injection).

The assessment of the individual may be performed at the end of the recommended treatment (e.g., 24 months) or the minimum treatment (e.g., 3 months for “low risk”, 6 months for “moderate risk”, or 12 months for “high risk”). The assessment may also be performed during or after the treatment. The individual may be subjected to a skin prick test. The individual may be determined as being non-allergic to the allergen if the skin prick test yields a negative result (e.g., a response no greater than 4 mm). The individual may be determined as being allergic to the allergen if the skin prick test yields a positive result (e.g., a response greater than 4 mm).

The individual may also be subjected to an oral food challenge. The oral food may include one or more of the administrated regimen. The individual may be determined as being non-allergic to the allergen if the oral food challenge results in no reaction. The individual may be determined as being allergic to the allergen if the oral food challenge yields any allergic response. The response may be observed in the form of one or more of the following signs: hives or other rashes, extremely pale skin, swelling of the skin, lips, or face, wheezing, or other severe breathing difficulties, sweating or dizziness, vomiting, diarrhea, or other abnormal gastrointestinal symptoms, bleeding, rapid pulse, fainting or loss of consciousness.

If desired, one may attempt to conduct pre-clinical studies in an animal model of allergy, although such models are not generally considered to be entirely reliable for predicting outcomes in human subjects. Nevertheless, the biology underlying allergen induced anaphylaxis may be explored in wild-type C57BL/6, B cell-deficient, CD40L-deficient, mast cell-deficient or FcεR1-chain-deficient mice sensitized to allergens. After intraperitoneal challenge with a formulation described herein, anaphylaxis can be assessed by measuring antigen-specific immunoglobulins (Igs), an overall symptom score, body temperature, vascular permeability, mast cell mediator release and anaphylactic reactions. The B cell-, mast cell- and CD40L-deficient mice may be sensitized to allergens as shown by the production of IgE and Th2-associated cytokines. The FcεR1-chain-deficient mice may experience anaphylaxis albeit somewhat less severe than the wild-type animals. In a model of esophago-gastro-enteropathy induced by long term feeding of peanuts to sensitized mice, epicutaneous immunotherapy with a formulation described herein may lessen the severity of gastro-intestinal lesions (see Mondoulet et al., 2012).

### EXAMPLES

### Example 1

Rationale for Selection of Study Drug Regimen: The rationale for the study is to demonstrate a reduction in risk of developing multiple allergies or in incidence after the introduction of multiple allergens early in an individual's life. Potential risks include incorrectly diagnosing an individual as allergy free upon entrance to the study, which could put them at risk for having a reaction during the course of treatment. The immediate benefits for the individual include a potential decrease in the risk profile for developing an allergy (e.g., to peanuts, shellfish, soy, wheat, and pet dander).

Primary objective: A primary objective is to demonstrate a reduction of risk to an individual or a lowered incidence of allergies through escalating doses of orally introduced proteins derived from multiple allergenic substances. Secondarily, the study can demonstrate the safety of the regimen, formulation, and delivery method as well as evaluate the immunological effects of the proposed method and composition.

Design: One-hundred (100) subjects who pass screening will be randomized 1:1 to active therapy or control. Individuals will escalate their doses up to an expected daily dose of 5 g of protein derived from allergenic substances in subjects able to tolerate up-dosing. Clinical data and blood samples will be collected at pre-specified intervals. If an individual is removed from therapy because of failing escalation or build-up, the individual will continue to be followed for safety. The therapy is comprised of two months of continued escalating exposure and new protein introductions to the multiple allergen formulation. Subjects not being able to tolerate any of the doses will be removed from the trial. A follow-up assessment is conducted at six months and incremental assessments are planned but not required at nine months, 12 months, two years, five years, and possibly ten years for longitudinal analysis. Those still enrolled after six months will be given an oral challenge that mimics normal exposure to multiple allergens. They will also have IgE levels checked and pin prick wheal size measured. For those in the control arm, they will be not be directly subjected to allergen exposure, but they may be recommended to ingest commercially available products in-line with their pediatrician's recommendations. They will only have pin prick wheal size measured and, optionally, a serum IgE test for reactivity to Ara h 1-9. All dose escalations will be supervised in the clinic, and dosing symptoms and adverse events will be captured throughout the study. A person who is carrying an epinephrine auto-injector and trained in its use will be present when the composition is administered.

Primary Efficacy Outcome Measure and Secondary Endpoints: The primary clinical efficacy end-point is the successful completion of an oral food challenge comprising of 5 g of protein derived from multiple allergenic substances. Other endpoints could include comparison mean wheal sizes, IgE levels against Ara h 1-9. The safety of the therapy is measured through dosing symptoms, adverse events and serious adverse events.

Study Product: The study product is characterized peanut allergen as peanut flour, characterized tree nuts as additional flours, pet dander as harvested protein, soy as an extract, shellfish as an extract, wheat as a flour or extract, vitamin D, and vitamin A, vitamin C, and vitamin E formulated with a bulking agent and a flow agent in pre-measured graduated doses, comprising single-use vials containing escalating doses of protein with established ratios of key allergens. The vials will be opened, rolled gently and tapped lightly to ensure full delivery of the contents, and mixed with food prior to ingestion by the individual. All study product will be packaged and labeled at the central manufacturer. Study drug will be shipped by the drug depot to the site pharmacist for distribution to the site study personnel. The site pharmacist will dispense the study drug in a manner consistent with the current dose level and treatment assignment. The product will be labeled with the numbered dose level and package number.

Preparation, Administration and Dosage: The drug product will be provided pre-packaged from the site pharmacy in appropriate doses to deliver the specified dose. The contents may be added to apple juice, applesauce, yogurt, pudding or other age-appropriate food. The food may not be heated before consumption, and must also be one to which the subject is not additionally allergic. The containers holding the compositions to be administered should be drawn apart in the event they are joined (e.g., by perforated packaging), and gently agitated (e.g., rolled between finger and thumb). Simple mechanics, such as a light tap, can help to ensure full delivery of the contents. The product is preferably consumed promptly after mixing. If there is a delay of more than 24 hours in consumption following mixing with a food or beverage, that final product may be discarded and a new product dose mixed and consumed. We anticipate a target interval of at least or about 12 hours between doses.

### Example 2

### Rationale for Selection of Study Drug Regimen

The rationale for the study is to demonstrate a reduction in risk of developing allergies or in incidence after the introduction of allergens early in an individual's life. Potential risks include incorrectly diagnosing an individual as allergy free upon entrance to the study, which could put them at risk for having a reaction during the course of treatment. The immediate benefits for the individual include a potential decrease in the risk profile for developing an allergy to peanuts.

Primary Objective:

A primary objective is to demonstrate a reduction of risk to an individual or a lowered incidence of peanut allergies through escalating doses of orally introduced proteins derived from peanut. Secondarily, the study can demonstrate the safety of the regimen, formulation, and delivery method as well as evaluate the immunological effects of the proposed method and composition. The study can also demonstrate the composition's ease of use, tendency to be used, and overall satisfaction provided as compared to currently available peanut products.

Design:

Fifty (50) subjects who pass screening will be randomized 1:1 to active therapy or control. Individuals will escalate their dose twice within one day, supervised by a physician, up to an expected daily dose of 1 g of peanut protein in subjects able to tolerate up-dosing. Clinical data and blood samples will be collected at pre-specified intervals. If an individual is removed from therapy because of failing escalation or build-up, the individual will continue to be followed for safety. The therapy is comprised of two months of continued exposure to the peanut formulation. Subjects not being able to tolerate any of the doses will be removed from the trial. A follow-up assessment is conducted at six months and incremental assessments are planned but not required at nine months, 12 months, two years, five years, and possibly ten years for longitudinal analysis. Those still enrolled after six months will be given an oral challenge that mimics normal exposure to peanut. They will also have IgE levels checked and pin prick wheal size measured. For those in the control arm, they will be not be directly subjected to peanut exposure, but they may be recommended to ingest commercially available products in-line with their pediatrician's recommendations. They will only have pin prick wheal size measured and, optionally, a serum IgE test for reactivity to Ara h 1-9. All dose escalations will be supervised in the clinic, and dosing symptoms and adverse events will be captured throughout the study.

A person who is carrying an epinephrine auto-injector and trained in its use will be present when the composition is administered.

Primary Efficacy Outcome Measure and Secondary Endpoints:

The primary clinical efficacy end-point is the successful completion of an oral food challenge comprising of 5 g of protein derived from peanut. Other endpoints could include comparison mean wheal sizes, IgE levels against Ara h 1-9 and whole peanut powder when contrasting the control and therapy arms of the trial, patient safety, patient adherence, and patient satisfaction. The safety of the therapy is measured through dosing symptoms, adverse events and serious adverse events.

Study Product:

The study product is characterized peanut allergen as peanut flour, vitamin D, and vitamin A, vitamin C, and vitamin E formulated with a bulking agent and a flow agent in pre-measured graduated doses, comprising single-use vials containing escalating doses of peanut protein with established ratios of key peanut allergens. The vials will be opened, rolled gently and tapped lightly to ensure full delivery of the contents, and mixed with food prior to ingestion by the individual. All study product will be packaged and labeled at the central manufacturer. Study drug will be shipped by the drug depot to the site pharmacist for distribution to the site study personnel. The site pharmacist will dispense the study drug in a manner consistent with the current dose level and treatment assignment. The product will be labeled with the numbered dose level and package number.

Preparation, Administration and Dosage:

The drug product will be provided pre-packaged from the site pharmacy in appropriate doses to deliver the specified dose. The contents may be added to apple juice, applesauce, yogurt, pudding or other age-appropriate food. The food may not be heated before consumption, and must also be one to which the subject is not additionally allergic. The containers holding the compositions to be administered should be drawn apart in the event they are joined (e.g., by perforated packaging), and gently agitated (e.g., rolled between finger and thumb). Simple mechanics, such as a light tap, can help to ensure full delivery of the contents. The product is preferably consumed promptly after mixing. If there is a delay of more than 24 hours in consumption following mixing with a food or beverage, that final product may be discarded and a new product dose mixed and consumed. We anticipate a target interval of at least or about 12 hours between doses.

### Example 3

### Preparation of a Composition for Allergy Prevention

A composition comprising one or more dosages of an admixture is prepared, wherein each dosage of the admixture includes the following:
A. a quantity of allergenic substance, wherein the quantity is defined as a target amount plus/minus a variance;
B. a vitamin supplement comprising two or more of:
   400 IU±20% vitamin D3;
   750 IU±20% vitamin A;
   35 mg±20% vitamin C;
   5 IU±20% vitamin E; and
C. a physiologically acceptable carrier (e.g., vegetable oil and/or rice oil).

Administration of a Composition for Allergy Prevention, Treatment Methodology

Unit dosages of the admixture are administered to one or more human infant subjects, whose ages range from 3-7 months at the time of the first administration. Unit dosages are administered to the subjects on at least a daily basis for a period of two (2) months. The subjects are observed and/or tested (e.g., as described elsewhere herein) routinely during this period for allergic reaction to the admixture and, absent such reaction, administration is continued for that period.

At the close of that period, the subjects are observed and/or tested (e.g., as described elsewhere herein) for allergic reaction to the allergen contained in the admixture. The efficacy of treatment using the admixture in accord with the foregoing methodology can be quantified by a comparative measure of the subjects who experience allergic reaction to the allergen after the treatment period in comparison to those in a similar cohort of untreated individuals who experience allergic reaction to the allergen.

In another test of efficacy, the foregoing methodology is applied to the same or other infant subjects on a weekly basis.

In another test of efficacy, any of the foregoing methodologies is applied to the same or other infant subjects on a regular basis, albeit not necessarily daily or weekly basis.

In another test of efficacy, any of the foregoing methodologies is applied to the same or other infant subjects for a period of 3 months.

In another test of efficacy, any of the foregoing methodologies is applied to the same or other infant subjects for a period of 6 months.

In another test of efficacy, any of the foregoing methodologies is applied to the same or other infant subjects for a period of 12 months.

In another test of efficacy, any of the foregoing methodologies is applied to the same or other infant subjects for a period of 24 months.

In another test of efficacy, any of the foregoing methodologies is applied to the same or other infant subjects using an admixture incorporating three or more of the vitamins of component (B).

In another test of efficacy, any of the foregoing methodologies is applied to the same or other infant subjects using an admixture incorporating all four of the vitamins of component (B).

### Example 3(a)

### Peanut

The methodologies discussed above under the heading “Example 2: Preparation and Administration of a Composition for Allergy Prevention Preparation” are practiced with peanut protein as the allergenic substance, with 5/7 g as the target amount and 20% as the variance.

The methodologies discussed above under the heading “Example 2: Preparation and Administration of a Composition for Allergy Prevention Preparation” are practiced with peanut protein as the allergenic substance, with a fixed value of between 5 mg-1 g as the target amount for each of the subjects, where that fixed value (and, therefore, target amount) varies randomly, incrementally or otherwise, as between different groups of one or more subjects, and 20% as the variance.

### Example 3(b)

### Shellfish

The methodologies discussed above under the heading “Example 2: Preparation and Administration of a Composition for Allergy Prevention Preparation” are practiced with shellfish protein as the allergenic substance, with 1 g as the target amount and 20% as the variance. The methodologies discussed above under the heading “Example 2: Preparation and Administration of a Composition for Allergy Prevention Preparation” are practiced with shellfish protein as the allergenic substance, with a fixed value of between 5 mg-1 g as the target amount for each of the subjects, where that fixed value (and, therefore, target amount) varies randomly, incrementally or otherwise, as between different groups of one or more subjects, and 20% as the variance.

### Example 3(c)

### Tree Nut

The methodologies discussed above under the heading “Example 2: Preparation and Administration of a Composition for Allergy Prevention Preparation” are practiced with tree nut protein as the allergenic substance, with 12/7 g as the target amount and 20% as the variance.

The methodologies discussed above under the heading “Example 2: Preparation and Administration of a Composition for Allergy Prevention Preparation” are practiced with tree nut protein as the allergenic substance, with a fixed value of between 5 mg-2 g as the target amount for each of the subjects, where that fixed value (and, therefore, target amount) varies randomly, incrementally or otherwise, as between different groups of one or more subjects, and 20% as the variance.

### Example 3(d)

### Soy

The methodologies discussed above under the heading “Example 2: Preparation and Administration of a Composition for Allergy Prevention Preparation” are practiced with soy protein as the allergenic substance, with 6/7 g as the target amount and 20% as the variance.

The methodologies discussed above under the heading “Example 2: Preparation and Administration of a Composition for Allergy Prevention Preparation” are practiced with soy protein as the allergenic substance, with a fixed value of between 5 mg-1 g as the target amount for each of the subjects, where that fixed value (and, therefore, target amount) varies randomly, incrementally or otherwise, as between different groups of one or more subjects, and 20% as the variance.

### Example 3(e)

### Wheat

The methodologies discussed above under the heading “Example 2: Preparation and Administration of a Composition for Allergy Prevention Preparation” are practiced with wheat protein as the allergenic substance, with 1 g as the target amount and 20% as the variance.

The methodologies discussed above under the heading “Example 2: Preparation and Administration of a Composition for Allergy Prevention Preparation” are practiced with wheat protein as the allergenic substance, with a fixed value of between 3 mg-1 g as the target amount for each of the subjects, where that fixed value (and, therefore, target amount) varies randomly, incrementally or otherwise, as between different groups of one or more subjects, and 20% as the variance.

### Example 3(f)

### Dairy

The methodologies discussed above under the heading “Example 2: Preparation and Administration of a Composition for Allergy Prevention Preparation” are practiced with dairy protein as the allergenic substance, with 1 g as the target amount and 20% as the variance.

The methodologies discussed above under the heading “Example 2: Preparation and Administration of a Composition for Allergy Prevention Preparation” are practiced with dairy protein as the allergenic substance, with a fixed value of between 3 mg-1 g as the target amount for each of the subjects, where that fixed value (and, therefore, target amount) varies randomly, incrementally or otherwise, as between different groups of one or more subjects, and 20% as the variance.

### Example 3(g)

### Egg

The methodologies discussed above under the heading “Example 2: Preparation and Administration of a Composition for Allergy Prevention Preparation” are practiced with egg protein as the allergenic substance, with 1 g as the target amount and 20% as the variance.

The methodologies discussed above under the heading “Example 2: Preparation and Administration of a Composition for Allergy Prevention Preparation” are practiced with egg protein as the allergenic substance, with a fixed value of between 3 mg-1 g as the target amount for each of the subjects, where that fixed value (and, therefore, target amount) varies randomly, incrementally or otherwise, as between different groups of one or more subjects, and 20% as the variance.

### Example 3(h)

### Multi-Allergen

The methodologies discussed above under the heading “Example 2: Preparation and Administration of a Composition for Allergy Prevention Preparation” are practiced with n (where n is greater than one) of the allergenic substances identified in Examples 3(a)-3(g), wherein each dosage of the admixture includes the respective allergenic substances in target amounts as listed in the respective example and description above. In one example, the allergenic substance(s) include one or more environmental allergens (e.g., pet dander, pollen, ragweed, and the like).

### CONCLUSION

Described above are compositions, methods of treatment and kits according to the invention. Those skilled in the art are will appreciate that the embodiments described herein are examples of the invention and that other embodiments making changes therein fall in the scope of the hereof, of which we claim:

1-145. (canceled)

146. A composition comprising one or more dosages of an admixture, wherein each dosage of the admixture includes the following:
A. two or more of:
i. a quantity of allergenic substance, wherein the quantity is defined as a target amount plus/minus a variance, wherein the allergenic substance is peanut protein, and wherein the target amount is 5 mg-1 g and the nce is 20%;
ii. a quantity of allergenic substance, wherein the quantity is defined as a target amount plus/minus a variance, wherein the allergenic substance is shellfish protein, and wherein the target amount is 5 mg-1 g and the variance is 20%;
iii. a quantity of allergenic substance, wherein the quantity is defined as a target amount plus/minus a variance, wherein the allergenic substance is tree nut protein, and wherein the target amount is 5 mg-2 g and the variance is 20%;
iv. a quantity of allergenic substance, wherein the quantity is defined as a target amount plus/minus a variance, wherein the allergenic substance is soy protein, and wherein the target amount is 5 mg-1 g and the variance is 20%;
v. a quantity of allergenic substance, wherein the quantity is defined as a target amount plus/minus a variance, wherein the allergenic substance is wheat protein, and wherein the target amount is 3 mg-1 g and the variance is 20%;
vi. a quantity of allergenic substance, wherein the quantity is defined as a target amount plus/minus a variance, wherein the allergenic substance is dairy protein, and wherein the target amount is 3 mg-1 g and the variance is 20%;
vii. a quantity of allergenic substance, wherein the quantity is defined as a target amount plus/minus a variance, wherein the allergenic substance is egg protein, and wherein the target amount is 3 mg-1 g and the variance is 20%;
B. a vitamin supplement comprising two or more of:
400 IU±20% vitamin D3;
750 IU±20% vitamin A;
35 mg±20% vitamin C;
5 IU±20% vitamin E; and
C. a physiologically acceptable carrier.

147-148. (canceled)

149. The composition of claim 146, wherein the composition comprises a single unit dose of the admixture.

150. The composition of claim 146, wherein each dosage comprises 50 microliters-20 mL of the admixture.

151. The composition of claim 146, wherein the vitamin supplement comprises all four of the vitamin D3, vitamin A, vitamin C, and vitamin E.

152. The composition of claim 146, wherein:
A. the composition comprises a single 50 microliters-20 mL unit dose of the admixture;
B. the variance is 5%;
C. component (B) comprises:
400 IU±5% vitamin D3,
750 IU±5% vitamin A,
35 mg±5% vitamin C,
5 IU±5% vitamin E; and
D. component (C) comprises a vegetable oil.

153. A method of allergy prevention comprising:
A. selecting a subject without an allergic reaction to two or more allergenic substances, wherein the two or more allergenic substances are selected from peanut protein, shellfish protein, tree nut protein, soy protein, wheat protein, dairy protein, and egg protein;
B. repeatedly administering a dosage of an admixture according to claim 146 to the subject, wherein the admixture comprises the two or more allergenic substances:
C. thereby, preventing or reducing a risk that the subject will develop an allergy to the allergenic substances.

154. The method of claim 153, wherein the selecting step includes selecting, as the subject, an infant.

155. The method of claim 153, wherein step (B) includes administering the dosage at least weekly.

156. The method of claim 153, wherein step (B) includes administering the dosage for at least six months.

157. The method of claim 153, wherein step (B) includes administering the dosage for at least twelve months.

158. The method of claim 153, wherein the variance is 5%.

159. The method of claim 153, wherein the admixture comprises 400 IU±5% vitamin D3, 750 IU±5% vitamin A, 35 mg±5% vitamin C, and 5 IU±5% vitamin E.

160. The method of claim 153, wherein the physiologically acceptable carrier comprises a vegetable oil.

161. The method of claim 153, wherein each dosage comprises 50 microliters-20 mL of the admixture.

162. The method of claim 153, wherein the vitamin supplement comprises three or four of the vitamin D3, vitamin A, vitamin C, and vitamin E.

163. The method of claim 153, wherein the dosage is administered orally.

164. The method of claim 153, wherein the individual has an IgE level against whole peanut or against one or more of Ara h 1-Ara h 9 of less than about 1 ku/L (2.4 ng/mL).

165. The method of claim 153, wherein the individual is negative for pin prick test as determined by a wheal size of less than about 4 mm in diameter.

166. The method of claim 153, wherein the individual is a newborn baby or infant under 18 months of age when the composition is first administered.

167. The method of claim 153, wherein the individual is an infant between 4 and 6 months of age when the composition is first administered.

168. A composition comprising one or more dosages of an admixture, wherein each dosage of the admixture includes the following:
A. a quantity of allergenic substance, wherein the quantity is defined as a target amount plus/minus a variance, wherein the allergenic substance is peanut protein, and wherein the target amount is 5 mg-1 g and the nce is 20%;
B. one or more of:
i. a quantity of allergenic substance, wherein the quantity is defined as a target amount plus/minus a variance, wherein the allergenic substance is shellfish protein, and wherein the target amount is 5 mg-1 g and the variance is 20%;
ii. a quantity of allergenic substance, wherein the quantity is defined as a target amount plus/minus a variance, wherein the allergenic substance is tree nut protein, and wherein the target amount is 5 mg-2 g and the variance is 20%;
iii. a quantity of allergenic substance, wherein the quantity is defined as a target amount plus/minus a variance, wherein the allergenic substance is soy protein, and wherein the target amount is 5 mg-1 g and the variance is 20%;
iv. a quantity of allergenic substance, wherein the quantity is defined as a target amount plus/minus a variance, wherein the allergenic substance is wheat protein, and wherein the target amount is 3 mg-1 g and the variance is 20%;
v. a quantity of allergenic substance, wherein the quantity is defined as a target amount plus/minus a variance, wherein the allergenic substance is dairy protein, and wherein the target amount is 3 mg-1 g and the variance is 20%;
vi. a quantity of allergenic substance, wherein the quantity is defined as a target amount plus/minus a variance, wherein the allergenic substance is egg protein, and wherein the target amount is 3 mg-1 g and the variance is 20%;
C. a vitamin supplement comprising two or more of:
400 IU±20% vitamin D3;
750 IU±20% vitamin A;
35 mg±20% vitamin C;
5 IU±20% vitamin E; and
D. a physiologically acceptable carrier.

169. A composition comprising one or more dosages of an admixture, wherein each dosage of the admixture includes the following:
A. a quantity of allergenic substance, wherein the quantity is defined as a target amount plus/minus a variance, wherein the allergenic substance is peanut protein, and wherein the target amount is 5 mg-1 g and the nce is 20%;
B. a quantity of allergenic substance, wherein the quantity is defined as a target amount plus/minus a variance, wherein the allergenic substance is tree nut protein, and wherein the target amount is 5 mg-2 g and the variance is 20%;
C. a vitamin supplement comprising two or more of:
400 IU±20% vitamin D3;
750 IU±20% vitamin A;
35 mg±20% vitamin C;
5 IU±20% vitamin E; and
D. a physiologically acceptable carrier.
